(19) United States
(12) Patent Application Publication
(10) Pub. No.: US 2014/0261450 A1
(43) Pub. Date: Sep. 18, 2014

(54) INTRAORAL MANDIBULAR ADVANCING POSITIVE PRESSURE APPARATUS

(71) Applicant: MICHAEL BRADY MOREHEAD, SAN ANTONIO, TX (US)

(72) Inventor: MICHAEL BRADY MOREHEAD, SAN ANTONIO, TX (US)

(21) Appl. No.: 14/207,054

(22) Filed: Mar. 12, 2014

Related U.S. Application Data
(60) Provisional application No. 61/800,330, filed on Mar. 15, 2013.

(51) Int. Cl. A61F 5/56; A61M 16/00; A61M 16/20; A61M 16/04; A61M 16/08

(52) U.S. Cl. CPC A61F 5/566; A61M 16/049; A61M 16/0875; A61M 16/0816; A61M 16/20; A61M 16/0057
USPC 128/848

(57) ABSTRACT

The intraoral mandibular advancing positive pressure apparatus utilizes mandibular advancement (or protrusion) therapy with CPAP therapy. The mandibular advancement appliance is fabricated to receive a portion of a person's upper and lower teeth when the patient bites down on the appliance. The mandibular advancement appliance advances the patient's mandible a predetermined amount. The mandibular advancement appliance contains a fluid communication pathway that provides for access to the patient's oral cavity. A CPAP device is in fluid communication with the fluid communication pathway and supplies a continuous positive pressure air supply. The utilization of mandibular advancement therapy with CPAP therapy reduces the amount of advancement necessary through mandibular advancement and eliminates the need for nasal involvement or a facial mask typically associated with CPAP therapy.

### CROSS-REFERENCES TO RELATED APPLICATIONS

This application claims the benefit of U.S. provisional application Ser. No. 61/800,330 filed Mar. 15, 2013 entitled Intraoral Mandibular Advancing Positive Pressure Device, which is incorporated by reference herein.

### STATEMENT REGARDING FEDERALLY SPONSORED RESEARCH OR DEVELOPMENT

Not applicable.

### BACKGROUND

1. Field.

The present apparatus and method relate to the treatment of obstructive sleep apnea (“OSA”).

2. Description of the Related Art.

OSA is a common disorder with some estimates ranging from 12 to 18 million people in the United States needing treatment. Others estimate that if the general public were studied in a sleep lab by polysomnography (formal “sleep study”), that approximately 1 in 5 American adults would have at least mild OSA. A S Shamsuzzaman, B J Gersh & V K Somers, Obstructive sleep apnea: implications for cardiac and vascular disease, 290 J. Am. Med. Ass'n. 1906-14 (2003). The most serious and deadly consequences of OSA are the cardiovascular diseases that can arise. Hypertension, tachycardia, increased risk of cerebrovascular accidents, daytime hypercapnia, atrial fibrillation, and even coronary artery disease all have been associated with OSA. Y Sharabi, Y Dagan & E Grossman, Sleep apnea as a risk factor for hypertension, 13 Current Opinion in Nephrology and Hypertension 359-64 (2004).

The general aim in treating sleep-breathing disorders is to facilitate breathing by increasing the airway passages. Different surgical and non-surgical treatments for OSA have emerged due to the varied causes of OSA. Treatments all focus on preventing collapse of the lumen of the pharynx during sleep.

Surgical treatment is generally reserved for patients with severe OSA or those that do not tolerate non-surgical treatments. One of the more common surgical treatments is orthognathic surgery where a patient's mandible, and/or maxilla, is moved forward. Moving the mandible forward causes the tongue and pharyngeal dilator muscles, and indirectly the soft palate, to move forward opening or expanding the oropharyngeal airways. Surgery is disfavored due to the aggressiveness of the surgery and the lengthy recovery time, often requiring the patient to adhere to an all liquid diet for six weeks.

Home use of a CPAP device is generally regarded as the gold standard for initial treatment of OSA. R Zozula & R Rosen, Compliance with continuous positive airway pressure therapy: Assessing and improving treatment outcomes, 7 Current Opinion in Pulmonary Med. 391-98, 2001. Treatment using a CPAP device has shown to be very effective in most OSA patients regardless of where the obstruction in the pharynx occurs. In this treatment method, the patient wears a mask at night that generally encompasses the mouth and nose. A machine continuously forces air through a hose that is connected to the mask. The forced air keeps the pharynx unobstructed, due solely to the increased air pressure, similar to as if it were being blown up like a balloon. The amount of air necessary to keep the pharynx unobstructed varies based on the patient and severity of the OSA, as a result, the CPAP machine may be regulated to determine the proper amount of forced air necessary. However, many patients have poor long term compliance because of the amount of pressure that is required as a sole source of oral pharyngeal patency.

While treatment through a CPAP device has been shown to be highly effective, there exist several negative aspects that reduce compliance. The most important aspect is that many patients simply cannot tolerate the treatment. Many complain of not being able to sleep with a mask on their face or that it feels unnatural to have air blown down their throat for extended times at a high air pressure. Other reasons for discontinuing CPAP therapy have been primarily related to issues of nasal dryness and congestion and difficulty adapting to the high pressure. Other patients have more cosmetic complaints such as not wanting to wear the mask that is generally secured through an elastic band against the back of the head, skin irritation on the face from the mask, and the discomfort of feeling excess heat against the patient's face due to the mask. Research shows poor long term compliance of this treatment modality.

For patients with mild-to-moderate OSA who cannot tolerate CPAP treatment, and where orthognathic surgery is a too aggressive treatment, mandibular advancement through a removable oral appliance is a suitable alternative. The mandibular advancement device (“MAD”) resembles orthognathic surgery in that the device advances the mandible forward increasing the oropharyngeal airway. G T Clark, D Arand, E Chung, et al., Effect of anterior mandibular positioning on obstructive sleep apnea, 147 Am. Rev. Respiratory Disease 624-29, 1993. Patients undergo a trial and error phase of treatment to determine the correct amount of advancement that provides the most OSA treatment. J A Haskell, J McCrillis, B Haskell, et al., Effects of Mandibular Advancement Device (MAD) on Airway Dimensions Assessed with Cone-Beam Computed Tomography, 13 Seminars in Orthodontics 132-38, 2009. Generally, most patients need mandibular advancement equivalent to seventy-five percent of the patient's maximum advancement. This amount of advancement is generally effective for eighty percent of patients.

Use of an MAD avoids many of the drawbacks associated with CPAP treatment but creates a different set of problems. Many patients that require the jaw to be advanced further than typical comfort level, and those at the seventy-five percent advancement threshold, develop musculoskeletal discomfort and temporomandibular joint (“TMJ”) discomfort. This is due to the extreme advancement required to have the desired lumen obstruction of the pharynx to achieve satisfactory OSA treatment. Other patients may see their teeth changing positions, including formation of gaps or overcrowding, that may interfere with the patient's normal or regular bite. This may ruin previous orthodontia alignment or cause a patient to need additional orthodontia.

### BRIEF SUMMARY

The intraoral mandibular advancing positive pressure apparatus and method utilizes a CPAP therapy with mandibular advancement therapy to treat OSA The apparatus comprises an intra-oral appliance fabricated to correspond to a patient's maxillary and mandibular teeth. The appliance is custom fabricated to protrude the patient's mandible a predetermined amount. The appliance is in fluid communication with a CPAP device. The fluid communication path allows air to access the oral cavity of the patient from the CPAP device. The appliance virtually seals the oral cavity to prevent leaking of air between a patient's teeth and cheeks as well as to prevent leaking of the positive pressure air outside the patient's oral cavity. A bi-directional valve with at least one vent is located along the fluid communication path between the CPAP and the appliance. The bi-directional valve and at least one vent allow the patient's expiratory air, should it travel though the oral cavity of the patient, to escape the system. The bi-directional valve is readily available on the open market place and commonly found on current CPAP Devices.

The method for treating OSA includes the steps of advancing a patient's mandible a predetermined amount and supplying continuous positive air pressure to the patient's oral cavity. One method of achieving mandible advancement involves fabrication of an intra-oral appliance that causes mandibular advancement when the appliance is inserted into a patient's mouth. The amount of mandibular advancement is generally less than seventy five percent of the maximum mandibular advancement of a specific patient. The appliance is inserted into the mouth of the patient with the patient biting down onto the appliance. A portion of the patient's upper and lower teeth match the impressions of the appliance and retain the appliance within the patient's mouth. When the appliance is inserted, the patient's mandible is protruded by the predetermined amount. The appliance is in fluid communication with a CPAP device wherein the CPAP device supplies a steady flow of positive air into the oral cavity of the patient. As the patient exhales through the oral cavity, the expiratory air exits the intraoral mandibular advancing positive pressure apparatus through the bi-directional valve and vents.

The utilization of mandibular advancement therapy in conjunction with CPAP therapy results in effective treatment of OSA without many of the negative side effects each therapy holds. The supply of continuous positive air pressure to a mandibular advancement appliance generally allows for less mandibular advancement. This apparatus allows for less than seventy-five percent of mandibular advancement and generally requires only forty to fifty percent of the patient's maximum mandibular advancement. The patient experiences more comfort with less mandibular advancement and is less likely to develop TMJ, teeth realignment, and/or changes to a patient's bite. Due to advancement of the mandible resulting in alleviating some obstruction of the pharynx, less air pressure from the CPAP device is generally necessary to accomplish the clinical goal of treating OSA. Further, the sealing effect of the appliance allows for the elimination of the nasal component of standard CPAP interfaces, eliminates the need for a mask, eliminates the need for a restraint of a mask, reduces the amount of physical items in contact with the patient's face, and eliminates the common discomfort felt by typical CPAP patients.

### DESCRIPTION OF THE DRAWINGS

FIG. 1 illustrates an angled top view of an embodiment of an intraoral mandibular advancing positive pressure appliance.

FIG. 2 is a partial cross-section view of an embodiment of an intraoral mandibular advancing positive pressure appliance.

FIG. 3 is a side profile view of an embodiment of an intraoral mandibular advancing positive pressure appliance engaged with a person's maxilla and mandible.

FIG. 4 is a front view of an embodiment of an intraoral mandibular advancing positive pressure appliance engaged with a person's maxilla and mandible.

FIG. 5 is a close up view of an embodiment of an intraoral mandibular advancing positive pressure apparatus in use by a person.

FIG. 6 is a view of an embodiment of an intraoral mandibular advancing positive pressure apparatus in use by a person.

### DETAILED DESCRIPTION

FIGS. 1 and 2 disclose the intraoral mandibular advancing positive pressure appliance 10 comprising a mouthpiece 20 and breathing tube connector 30. The mouthpiece 20 comprises a maxillary portion 21, mandibular portion 23, sealing portion 25, and molding support 28. Maxillary portion 21 is generally shaped in the form of an arch and contains impressions of a portion of a patient's upper teeth 22. Mandibular portion 23 is generally shaped in the form of an arch and contains impressions of a portion of a patient's lower teeth 24 (as seen in FIG. 3). Molding support 28 is generally arch shaped contains openings 29 and is positioned between maxillary portion 21 and mandibular portion 23. Sealing portion 25 connects maxillary portion 21 and mandibular portion 23 through the openings 29 of the molding support 28. Sealing portion 25 connects maxillary portion 21 and mandibular portion 23 to create a seal between maxillary portion 21 and mandibular portion 23. The maxillary portion 21, mandibular portion 23, and sealing portion 25 are generally constructed of a pliable material such as polymethyl methacrylate (PMMA). Molding support 28 is more rigid than maxillary portion 21, mandibular portion 23, and sealing portion 25 and is generally constructed of copolyester. The position of the maxillary portion 21 relative to the mandibular portion 23 is predetermined based on the desired mandibular advancement for the patient.

Breathing tube connector 30 is generally oval shaped having a sidewall 31, exterior sidewall surface 32, interior sidewall surface 33, intraoral opening 34, and extra oral opening 35. Breathing tube connector 30 is fused to the molding support 28. Breathing tube connector 30 is positioned within in the sealing portion 25 proximal to the apex of the arch shaped sealing portion 25 between the maxillary portion 21 and mandibular portion 23, specifically between the impressions of the upper anterior teeth 26 and the lower anterior teeth 27 (as seen in FIG. 4). Breathing tube connector 30 extends away from the curve of the sealing portion 25. The position of breathing tube connector 30 within the sealing portion 25 creates a fluid communication pathway into the interior space 11 of mouthpiece 20.

As seen in FIG. 3, sidewall 31 further comprises a tapered portion 37. The tapered portion 37 consists of exterior sidewall surface 32 and interior sidewall surface 33 narrowing from the widest formation located proximal to the extra oral opening 35 to the narrowest portion located approximately in the middle of breathing tube connector 30. Ridge 36 is located proximal to extra oral opening 35 and circumscribes sidewall 31. Breathing tube connector 30 angles negatively, greater than zero degrees but less than ninety degrees, in relation to the center longitudinal axis of the intraoral opening 34.

Referring to FIGS. 3 and 4, a patient's upper teeth 40, as part of maxilla 41, correspond to the maxillary impressions 22 of maxillary portion 21. The patient's lower teeth 42, as part of the mandible 43, correspond to the mandibular impressions 24 of the mandibular portion 23. When the patient bites down on the mouthpiece 20, the patient's mandible and maxilla are held in place by the teeth in the respective impressions. The fixed position of the mandibular portion 23 causes the patient's mandible 43 to protrude relative to the maxilla 41 in the direction of arrow 44.

Referring to FIGS. 5 and 6, the mouthpiece 20 of the appliance 10 is located within the mouth of the patient. The patient's lips 45 rest on the tapered portion 37 of the breathing tube connector 30. The patient's upper teeth are engaged to the impressions of the maxillary portion and the lower teeth are engaged to the impressions of the mandibular portion. When a patient's teeth are engaged in the impressions, the fluid communication pathway created by the breathing tube connector is the only access into the oral cavity. The mouthpiece forms a virtual air tight seal keeping air from leaking between the maxillary portion 21 and mandibular portion 23.

Elbow connector 50 is a generally hollow tube having a sidewall 51, breathing tube connection opening 52, and a breathing hose connection opening 53. The elbow connector 50 has a straight portion 54 and an angled portion 55. The straight portion terminates at the breathing tube connection opening 52 and the angled portion 55 terminates at the breathing hose connection opening 53. The straight portion 54 and angled portion 55 meet at an approximate ninety degree angle resulting in the breathing hose connection opening 53 being oriented ninety degrees from the breathing tube connection opening 52. The breathing tube connection opening 52 is frictionally engaged to the exterior sidewall surface 32 of the breathing tube connector 30. Ridge 36 provides an abutment point for the elbow connector 50. A bi directional valve 56 is located within the angled portion 55. One or more air evacuation holes 57 are dispersed around the elbow connector 50 proximal to the bi-directional valve 56 and placed between the breathing tube connector 30 and the bi-directional valve 56. Hose 58 is frictionally engaged to the breathing hose connection opening 53. The hose 58 is connected to a standard CPAP device 59. Hose 58 is generally constructed of flexible material. Elbow connector 50 is generally constructed of light weight but rigid material. In another embodiment, the elbow connector 50 may contain a swivel joint allowing the elbow connector 50 to rotate around the exterior sidewall surface 32 of the breathing tube connector 30.

Fabrication of the appliance starts with the creation of impressions from a patient's maxillary and mandibular arches utilizing standard industry techniques. Using a George Gauge, or similar device, a patient's range of mandibular advancement is measured. For this treatment methodology, the amount of mandibular advancement for the patient is calculated at seventy-five percent or less of the patient's maximum mandibular advancement.

Stone models are created from the patient's impressions. The maxillary portion 21 and mandibular portion 23 are created from the stone models using industry standard techniques. The stone models are mounted to a dental articulator where the predetermined mandibular advancement is transferred to the dental articulator. The fabricated maxillary portion 21 and mandibular portion 23 are placed on the stone models with the mandibular stone model appropriately protruded. The vertical dimension of the occlusion is opened allowing insertion of the molded support 28 with the fused breathing tube connector 30 between the maxillary portion 21 and mandibular portion 23. Sealing portion 25 is created through PMMA resin which secures the maxillary portion 21 and mandibular portion 23 to the molded support 28. Openings 29 in the molded support 28 allow PMMA resin to create solid bond between molded support 28, maxillary portion 21, and mandibular portion 23 through sealing portion 25.

The method of treatment using the appliance 10 is disclosed in reference to FIGS. 3, 5, and 6. The mouthpiece 20 is inserted into the patient's mouth such that the patient bites or clamps down on the mouthpiece 20. The patient's upper teeth 40 engage the maxillary portion 21 and the patient's lower teeth 42 engage the mandibular portion 23. When the patient bites down, the patient's mandible 43 protrudes forward a predetermined amount based on the desired prescription.

The patient's lips 45 rest on the tapered portion 37 of the breathing tube connector 30. The breathing tube connection opening 52 of the elbow connector 50 is connected to the breathing tube connector 30 of appliance 10. The angle of the breathing tube connector 30 provides a more comfortable fit for the patient by reducing the weight applied to the appliance 10 and held in place by the patient's bite. The hose 58 connects to the breathing hose connection opening 53 on one end and connects to the CPAP device 59 on the other. A fluid communication pathway is created from the CPAP device 59 to the patient's oral cavity. Advancing the lower mandible increases the airway space by bringing the structures that make up the lumen of the oropharynx forward.

The CPAP device 59 is activated and set to a predetermined pressure setting. The CPAP device 59 forces air through the machine to the oral cavity of the patient through the fluid communication pathway comprising the hose 58, elbow connector 50, and appliance 10. The patient's natural bite keeps the appliance 10 in place. The sealing portion 25 of the appliance 10 and the patient's lips resting on the tapered sidewall 37 virtually seal the oral cavity to prevent escape of the positive pressure air and reduce the amount of positive air existing between the patient's teeth and cheeks. As the patient exhales, the expiratory air leaves the oral cavity, through the intra oral opening 34, into the connector tub 30, out the extra-oral opening 35 and to the elbow connector 56. The bi-directional valve 56 causes the expiratory air to exit the fluid communication path through the air evacuation holes 57. The positive pressure air keeps the pharynx unobstructed. The use of the appliance 10 reduces the amount of air necessary from the CPAP device 59 and makes it unnecessary to utilize a full face mask or require the blockage of nasal passages.

The present disclosure is described above in terms of a preferred illustrative embodiment of a specifically-described Intraoral Mandibular Advancing Positive Pressure Appliance. Those skilled in the art will recognize that alternative constructions of such an appliance may be used. Other aspects, features, and advantages may be obtained from a study of this disclosure and the drawings, along with the appended claims

I claim:

1. An apparatus for treating breathing-related sleep disorders comprising a mandibular advancement appliance having an inlet, a positive pressure air flow generator, and a first fluid communication pathway between said inlet and said air flow generator.

2. The apparatus according to claim 1 wherein said mandibular advancement appliance comprises an upper portion containing impressions of at least some of a person's maxillary teeth; a lower portion containing impressions of at least some of said person's mandibular teeth; an outlet positioned within said person's oral cavity; and a second fluid communication pathway between said inlet and said outlet.

3. The apparatus according to claim 2 wherein said lower portion is fixed in a forward position relative to said upper portion to advance said person's mandible.

4. The apparatus according to claim 3 wherein said second fluid communication pathway comprises a connector tube having a sidewall, a first end having said inlet, and a second end having said outlet.

5. The apparatus according to claim 4 wherein a first portion of said sidewall is a concave cylinder.

6. The apparatus according to claim 5 wherein longitudinal center line of said first portion of said connector tube is angled relative to the longitudinal center line of second portion of said connector tube.

7. The apparatus according to claim 4 wherein said first fluid communication path comprises a tube.

8. The apparatus according to claim 7 wherein said first fluid communication path further comprises a neck connector having a sidewall, a first portion connected to said tube, and a second portion connected to said first end of said connector tube.

9. The apparatus according to claim 8 wherein said first portion of said neck connector is angled relative to said second portion of said neck connector.

10. The apparatus according to claim 8 wherein said first portion of said neck connector is rotatable relative to said second portion of said neck connector.

11. The apparatus according to claim 8 wherein said neck connector contains a bi-directional valve.

12. The apparatus according to claim 11 wherein said neck connector further comprises at least one opening disposed through said sidewall.

13. The apparatus according to claim 2 wherein said upper portion is a “U”-shaped arch.

14. The apparatus according to claim 2 wherein said lower portion is a “U”-shaped arch.

15. The apparatus according to claim 2 wherein said mandibular advancement mouthpiece is integral.

16. A method for treating breathing-related sleep disorders comprising the steps of:
fabricating a mandibular advancement appliance comprising an inlet, outlet, and a lower portion fixed in a position to maintain forward advancement of a person's mandible;
placing said mandibular advancement appliance in said person's oral cavity wherein said outlet is located within said person's oral cavity and in fluid communication with said inlet; and
supplying continuous positive airway pressure through a fluid communication path to said inlet of said mandibular advancement appliance.

17. A method of claim 16 wherein said fluid communication path contains a bi-directional valve and at least one vent.

18. A method of claim 16 further comprises the step of measuring the maximum forward advancement of said person's mandible and fabricating said mandibular advancement appliance to correspond to seventy five percent or less of said maximum forward advancement.

19. A method of claim 16 wherein said continuous positive airway pressure is 14 cmH₂O or less.

20. A method for treating breathing related sleep disorders comprising the steps of:
advancing a patient's mandible forward; and
supplying continuous positive air pressure to a patient's oral cavity.
